(19) United States
(12) Patent Application Publication
(10) Pub. No.: US 2004/0132199 A1
(43) Pub. Date: Jul. 8, 2004

(54) Method for monitoring a reaction by coloring reaction in the solid phase synthesis of a sugar chain

(75) Inventors: Shino Manabe, Wako-shi (JP); Yukishige Ito, Wako-shi (JP)

Correspondence Address: GREENBLUM & BERNSTEIN, P.L.C., 1950 ROLAND CLARKE PLACE, RESTON, VA, 20191, US

(73) Assignee: RIKEN, Saitama (JP)

(21) Appl. No.: 10/673,131

(22) Filed: Sep. 30, 2003

(30) Foreign Application Priority Data
Oct. 28, 2002 (JP) 2002-312131

(51) Int. Cl. G01N 33/00

(52) U.S. Cl. 436/94

(57) ABSTRACT

An object of the present invention is to provide a method for simply, quickly and selectively monitoring the progress of a reaction with high sensitivity in real time in the solid-phase synthesis of a sugar chain. The present invention provides a method for detecting whether or not a hydroxyl group in sugars is protected, which comprises the step of reacting the sugar having a hydroxyl group or hydroxyl group protected by a Z—CH₂—CO— group wherein Z represents a halogen or —O—SO₂—R, in which R represents an aliphatic or aromatic hydrocarbon group, which is immobilized to a solid phase, with a compound represented by the formula X-Y wherein X represents a residue of an azo dye compound, and Y represents a group capable of reacting with the hydroxyl group in the sugars; and/or reacting the above sugar with (p-nitrobenzyl)pyridine under basic conditions.

### TECHNICAL FIELD

The present invention relates to a method for monitoring a reaction by coloring reaction in the solid phase synthesis of a sugar chain. More particularly, the present invention relates to a method for monitoring a reaction in the solid phase synthesis of a sugar chain wherein the presences of a hydroxyl group and a chloroacetyl group which is a protecting group of a hydroxyl group are detected by a coloring reaction.

### BACKGROUND ART

Conventionally, in order to confirm the progress of a solid-phase reaction of a sugar chain, a reaction product has been cleaved from the solid phase, or equipment such as a Magic angle spinning NMR has been required (Seeberger, P. H. et al., Angew. Chem. Int. Ed., 1997, 36,491-493). A method of incorporating a carbon isotope ¹³C or fluorine atom which can be measured by NMR into a protecting group and carrying out the measurement, is also known (Kanemitsu, T. et al., Angew. Chem. Int. Ed., 1998, 37, 3415-3418; Mogemark, M. et al., Org. Lett., 2001, 3, 1463-1466). However, these methods have disadvantages in that they require an expenditure of time and effort, or that they require special equipment and the reaction must be stopped for every measurement.

With regard to the solid-phase synthesis of a peptide, a technique of monitoring and confirming the progress of the reaction by a ninhydrin reaction that is a coloring reaction by an amino group which occurs between the amino group and ninhydrin, has been established (Kaiser, E. et al., Anal. Biochem., 1970, 34, 595-598). This technique is being applied to automated synthesizers.

With regard to the solid-phase synthesis of a sugar chain, however, since a coloring method similar to the above method of using an amino group in peptide synthesis has not been developed for a hydroxyl group in the main functional group, a method for monitoring a reaction using a simple coloring reaction has not yet been developed.

### DISCLOSURE OF THE INVENTION

Solid-phase synthesis is a key technique for automatic synthesis, and it is also a basic technique for combinatorial chemistry. As stated above, the solid-phase reaction has an advantage in that an excessive reagent or substrate can be washed away, but it has also a disadvantage in that monitoring of the reaction is extremely difficult. An object of the present invention is to provide a method for simply, quickly and selectively monitoring the progress of a reaction with high sensitivity in real time in the solid-phase synthesis of a sugar chain.

As a result of intensive studies directed towards the above object, the present inventors have succeeded in developing a technique for monitoring the progress of a reaction in the solid-phase synthesis of a sugar chain in a real-time manner. In the solid-phase synthesis of a sugar chain which comprises a repeat of two reactions of a glycosylation reaction and a deprotecting reaction of a temporary protecting group, a coloring reaction is necessary not only for a hydroxyl group but also for the temporary protecting group. Thus, the present inventors have detected a chloroacetyl group which is widely used as a temporary protecting group in the sugar chain synthesis by reacting the chloroacetyl group with (p-nitrobenzyl)pyridine (PNBP) (Compound 1 in the reaction scheme below) to develop red color under basic conditions. They have also detected a hydroxyl group using a Disperse Red (red dye)-cyanuric chloride complex (Compound 2 in the reaction scheme below). Thus, they have succeeded in monitoring the progress of a reaction in the solid-phase synthesis of a sugar chain in a real-time manner.

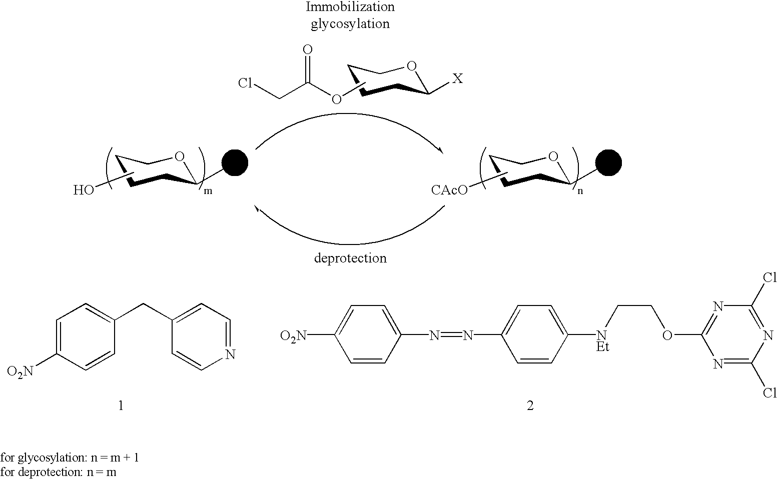

When a glycosylation reaction is carried out by using a sugar donor having a protecting group of a hydroxyl group such as a chloroacetyl group, the progress of the glycosylation reaction can be simply monitored by elimination of the hydroxyl group, that is, disappearance of the coloring reaction with Disperse Red and increase of the coloring reaction with PNBP. The deprotecting reaction of the protecting group of a hydroxyl group such as a chloroacetyl group can be simply monitored by disappearance of the coloring reaction with PNBP and increase of the coloring reaction with Disperse Red. Thus, according to the method of the present invention, the solid-phase reaction can be quickly monitored in real time, without cleaving a substrate from the solid phase, and without using special equipment. Also, only a very small amount of solid phase resin is required.

Thus, according to the present invention, there is provided a method for detecting the presence of a hydroxyl group in sugars, which comprises reacting the sugar having a hydroxyl group which is immobilized to a solid phase, with a compound represented by the formula X-Y wherein X represents a residue of an azo dye compound, and Y represents a group capable of reacting with the hydroxyl group of the sugars.

In another aspect of the present invention, there is provided a method for detecting the presence of a protected hydroxyl group in sugars, which comprises reacting the sugar having a hydroxyl group protected by a Z—CH₂CO— group wherein Z represents a halogen or —O—SO₂—R, in which R represents an aliphatic or aromatic hydrocarbon group, which is immobilized to a solid phase, with (p-nitrobenzyl)pyridine under basic conditions.

In another aspect of the present invention, there is provided a method for detecting whether or not a hydroxyl group in sugars is protected, which comprises the step of reacting the sugar having a hydroxyl group or hydroxyl group protected by a Z—CH₂—CO— group wherein Z represents a halogen or —O—SO₂—R, in which R represents an aliphatic or aromatic hydrocarbon group, which is immobilized to a solid phase, with a compound represented by the formula X-Y wherein X represents a residue of an azo dye compound, and Y represents a group capable of reacting with the hydroxyl group in the sugars; and/or reacting the above sugar with (p-nitrobenzyl)pyridine under basic conditions.

The compound represented by the formula X-Y is preferably N-[2-[(4,6-dichloro-1,3,5-triazin-2-yl)oxy]ethyl]-N-ethyl-4-[(4nitrophenyl)azo]-benzeneamide.

The Z—CH₂—CO— group is preferably a chloroacetyl group.

In another aspect of the present invention, there is provided a method for monitoring the progress of a synthesis reaction of a sugar chain in the method of synthesizing a sugar chain by reacting the first sugars having a hydroxyl group which is immobilized to a solid phase, with the second sugars having a reactive group reacting with the above hydroxyl group and a protected hydroxyl group, wherein the protecting group of the hydroxyl group is a Z—CH₂—CO— group wherein Z represents a halogen or —O—SO₂—R, in which R represents an aliphatic or aromatic hydrocarbon group, and the presence of a hydroxyl group or a protected hydroxyl group in sugars which is immobilized to a solid phase is detected by the reaction of the sugar with a compound represented by the formula X-Y wherein X represents a residue of an azo dye compound, and Y represents a group capable of reacting with the hydroxyl group in sugars, or (p-nitrobenzyl)pyridine.

### BRIEF DESCRIPTION OF THE DRAWINGS

FIG. 1 is a result obtained by monitoring the progress of the synthesis of a sugar chain which comprises the deprotection of a hydroxyl group by means of the method of the present invention.

### DETAILED DESCRIPTION OF THE INVENTION

The embodiments of the present invention will be described in detail below.

The present invention relates to a method for detecting the presence of a hydroxyl group or a protected hydroxyl group in sugars which are immobilized to a solid phase.

A hydroxyl group in sugars can be detected by reacting the sugar with a compound represented by the formula X-Y wherein X represents a residue of an azo dye compound, and Y represents a group capable of reacting with the hydroxyl group in sugars.

The compound represented by the formula X-Y wherein X represents a residue of an azo dye compound, and Y represents a group capable of reacting with the hydroxyl group in sugars, which is used to detect the hydroxyl group, is explained below.

The compound represented by the formula X-Y is colored because it contains an azo dye component represented by X. Moreover, this compound is able to bind to a hydroxyl group in sugars by means of a group represented by Y. The compound represented by the formula X-Y is immobilized to a solid phase by reacting sugars having a hydroxyl group which is immobilized to a solid phase with the compound of the formula X-Y. Then, the presence or absence of a hydroxyl group in sugars can be detected by determining the presence or absence of the coloration of the solid phase.

Specific examples of the azo dye component represented by X include: Disperse Red (common name): 2-[ethyl[4-[(4-nitrophenyl)azo]phenyl]amino)-ethanol;

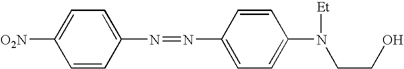

Basic Blue 41 (common name); 2-[4-[ethyl(2-hydroxyethyl)amino]phenyl]azo]-6-methoxy-3-methyl-benzothiazolium methyl sulfate;

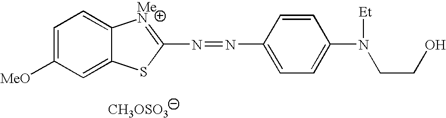

Congo Red (common name); 1-[[3-methyl-4-[(3-methylphenyl)azo]phenyl]azo-naphthanol;

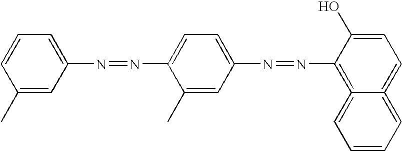

Sudan Red 7B (common name); N-ethyl 1-[[4-phenylazo)-phenylazo]-2-naphthaleneamine;

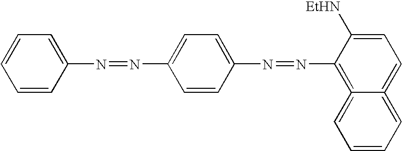

Congo Red (common name); 3,3′-[[1,1′-biphenyl-]-4,4′-diylbis(azo)-]bis[4-amino]-1-naphthalene sulfonic acid disodium salt; and

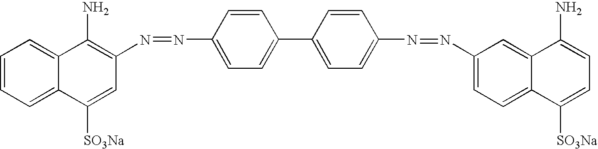

5-amino-3-[(1E)-phenylazo]-4-hydroxy-2,7-naphthalen disulfonic acid disodium salt;

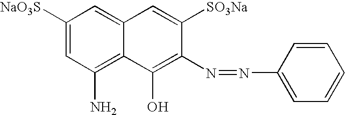

Specific examples of the group represented by Y which is capable of reacting with a hydroxyl group in sugars include a halogen; O—SO₂—R wherein R represents an aliphatic or aromatic hydrocarbon group; isothiocyanate, and a chlorotriazine derivative.

Specific examples of the preferred compound represented by the formula X-Y include: N-[2-[(4,6-dichloro-1,3,5-triazin-2-yl)oxy]ethyl]-N-ethyl-4-[(4-nitrophenyl)azo]-benzeneamide;

Procion Red MX-5B (common name); 5-[(4,6-dichloro-1,3,5-triazin-2-yl)amino]-4hydroxy-3-phenylazo 2,7-naphthalene disulfonic acid disodium salt; and

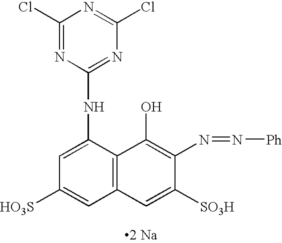

Cibacron Brilliant Red MX-5B (common name); 5-(benzoylamino)-3-[[(5-[[4-chloro-6-[(4-sulfophenyl)amino]-1,3,5-triazin-2-yl]amino]-2-sulfophenyl]azo]-4-hydroxy-2,7-naphthalene disulfonate disodium salt

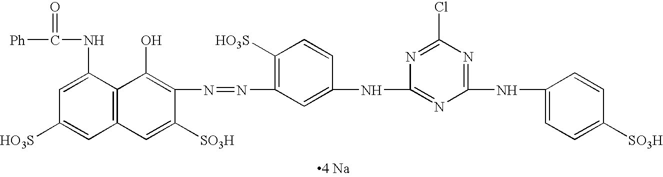

Moreover, a hydroxyl group protected by a Z—CH₂—CO— group in sugars can be detected by reacting the sugar with (p-nitrobenzyl)pyridine under basic conditions.

In the above formula, Z represents a halogen or —O—SO₂—R, wherein R represents an aliphatic or aromatic hydrocarbon group. Examples of halogen may include fluorine, chlorine, bromine and iodine, and chlorine is preferable. Moreover, examples of the aliphatic or aromatic hydrocarbon group represented by R include lower alkyl groups (e.g., those containing 1 to 6 carbon atoms) such as methyl, ethyl, propyl or butyl, aryl groups containing 6 to 12 carbon atoms such as phenyl or naphthyl, and alkyl groups obtained by their combination. A chloroacetyl group is a particularly preferred example of the Z—CH₂—CO— group.

Coloring reaction takes place by reacting a sugar having a hydroxyl group protected by a Z—CH₂—CO— group wherein Z represents a halogen or —O—SO₂—R, in which R represents an aliphatic or aromatic hydrocarbon group, which is immobilized to a solid phase, with (p-nitrobenzyl)pyridine under basic conditions, The term “under basic conditions” used herein mean that a reaction is carried-out preferably in the presence of a weak base such as pyridine, amines (e.g., N,N′-diisopropylamine, triethylamine, N-methylmorpholine, etc.), or anilines (e.g., aniline, N-methylaniline, p-toluidine). The presence of the hydroxyl group protected by the Z—CH₂—CO— group can be detected by determining the presence or absence of the development of a color.

The progress of the synthesis reaction of a sugar chain can be monitored by using the method of the present invention as mentioned above. The synthesis of a sugar chain is carried out by reacting the second sugar with the first sugar which is immobilized to a solid phase. The elongation reaction of a sugar chain can be carried out using a common method that is known to a person skilled in the art

The first sugar which is immobilized to a solid phase has a hydroxyl group which may have a protecting group as necessary. It is necessary to eliminate (i.e., deprotect) this protecting group of a hydroxyl group, as appropriate, for the following reaction with the second sugar. In the present invention, a Z—CH₂CO— group wherein Z represents a halogen or —O—SO₂—R, in which R represents an aliphatic or aromatic hydrocarbon group, is used as a protecting group of a hydroxyl group. As stated above, the presence of this protecting group can be detected by carrying out a coloring reaction of the sugar with (p-nitrobenzyl)pyridine under basic conditions. Moreover, a hydroxyl group from which its protecting group has been eliminated (deprotected) can be detected by a reaction of the sugar with a compound represented by the formula X-Y wherein X represents a residue of an azo dye compound, and Y represents a group capable of reacting with the hydroxyl group in sugars. In the present invention, the state of the protection and deprotection of the hydroxyl group can be monitored by these two types of reactions, and thereby the progress of the elongation reaction of a sugar chain can be monitored.

A solid phase used in the present invention is not particularly limited, and any solid phase which is used for the common solid-phase synthesis of a sugar chain can be used, as long as a sugar can be immobilized and operations such as washing and separation can be carried out. Specific examples of the solid phase carrier used in the present invention include a microplate, beads, a tube, a membrane, gel, and a micro-particle solid phase carrier (e.g., agarose particles, gelatin particles, kaoline particles, and synthetic polymer particles (latex particles, etc.)). Of these, a micro-particle solid phase carrier is preferably used.

The types of sugars that can be used in the present invention are not particularly limited, and any of monosaccharide, disaccharide, and polysaccharide such as trisaccharide may be used. Specific examples of such sugars include tetraose such as erythrose or threose; pentose such as ribose, arabinose or xylose; hexose such as glucose, mannose, galactose, allose or talose; sugars obtained by deoxylation of a part of these sugars, such as 2-deoxyglucose or 2-deoxyribose; and amino sugar such as 2-acetamido-2-deoxyglucose; oligosaccharide obtained by ether binding of the aforementioned sugars, such as lactose or chitobiose; sialic acid; and glucuronic acid. Furthermore, these sugars may exist as a D form and an L form. Both of these forms and their mixture may also be used.

The present invention will be further described in the following examples. The present invention is not limited by these examples.

### EXAMPLES

### (1) Synthesis of N-[2-[(4,6-dichloro-1,3,5-triazin-2-yl)oxy]ethyl]-N-ethyl4-((4-nitrophenyl)azo]-benzeneamide

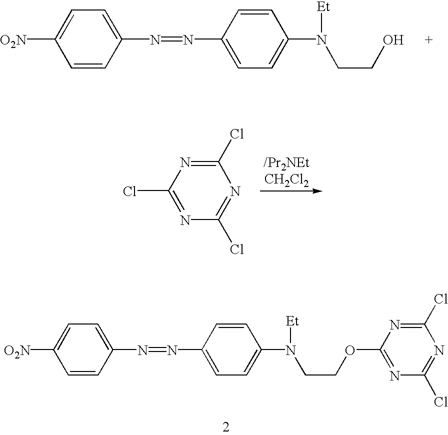

Disperse Red I (2.00 g, 6.36 mmol) and N,N′-diisopropylethylamine (1.1 mL, 6.36 mmol) were dissolved in CH₂Cl₂ (50 ml), and cyanuric chloride (1.17 g, 6.36 mmol, lachrymator) was added thereto at 0° C. The reaction solution was stirred at room temperature overnight. The solution was diluted with ethyl acetate (100 mL), and the organic layer was then washed with a cold saturated brine solution (30 mL). The water layer was extracted with ethyl acetate (30 mL), and the combined organic extracts were dried over sodium sulfate. After filtration, the solvent was subjected to vacuum concentration, and the residue was purified by silica gel column chromatography (CHCl₃: EtOAc was from 9:1 to 1:1), so as to obtain a product of interest (2.05 g, 70%). 1H NMR δ 8.34(dd, J=9.2 Hz, 2H), 8.0-7.9(m, 3H), 6.87(d, J=9.2Hz, 2H), 4.71(t, J=6.4Hz, 2H), 3.89(t, J=6.0 Hz, 2H), 3.62(q, J=7.6 Hz, 2H), 1.26(t, J=7.6 Hz, 3H).

### (2) Typical Experimental Method for Developing Color of Chloroacetyl Group by PNBP

Several particles of resin present in the reaction solution were transferred to a microtube, and approximately 0.3 mL of toluene solution of (p-nitrobenzyl)pyridine (80 mg/10 mL) was added thereto. After 5 minutes, the solution containing a solid phase resin was spread on a silica gel thin layer plate, and it was heated on a hot plate for 1 minute. Then, 10% piperidine solution in toluene was sprayed thereon followed by heating on the hot plate for 10 seconds. The color of the resin was visually observed by the naked eye, and it was also possible to capture its image into a computer, using a microscope equipped with a CCD camera (280×, CCD Micro Scope Inf-550 (Moritex)).

### (3) Typical Experiment Regarding Hydroxyl Group Coloring Method Using Disperse Red-cyanuric Chloride Complex

Several particles of resin present in the reaction solution were transferred to a microtube, and the particles were swelled with THF (0.1 mL). 1% Disperse Red solution in THF (0.1 mL), and one drop of 0.5% i-Pr₂NEt solution in THF were added thereto, and the resultant was left stand for 10 minutes, After 10 minutes, the supernatant was discarded, and the solid phase resin was washed with THF and DMF until the supernatant became transparent. The color of the resin was visually observed by the naked eye, and it was also possible to capture its image into a computer, using a microscope equipped with a CCD camera.

### Monitoring of Synthesis of Sugar Chain

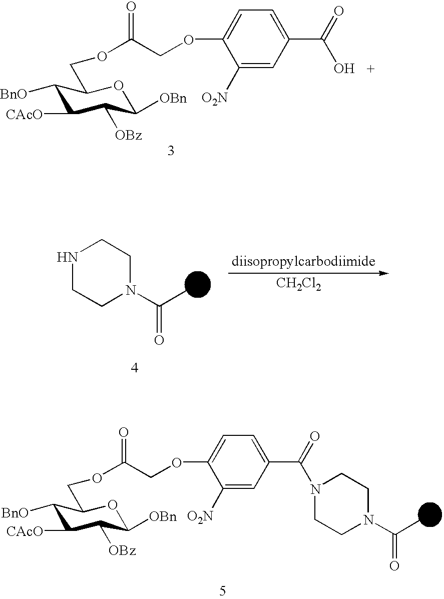

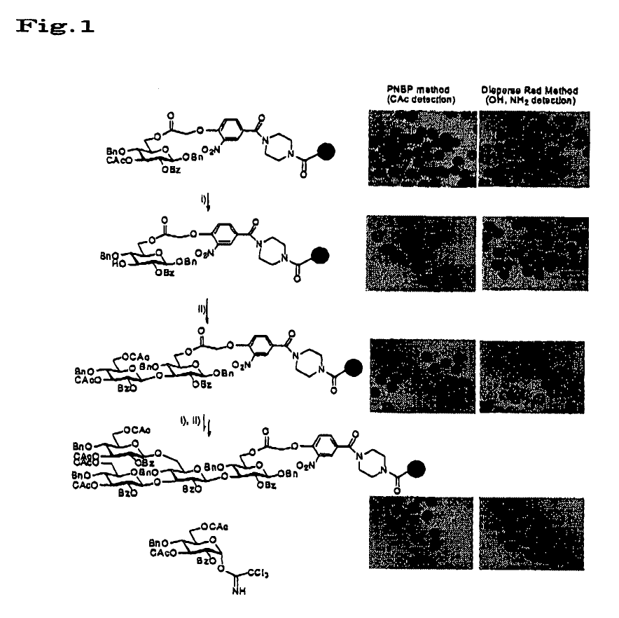

1,3-diisopropyl carbodiimide (88 μL, 0.563 mmol) was dropped at room temperature into a suspension (10 mL) of carboxylic acid 3 (411.0 mg, 0.563 mmol) and Tentagel type solid phase resin 4 (1.50 g, 1.375 mmol) in methylene chloride. The reaction suspension was shaken overnight, and the solid phase resin was filtrated. The solid phase resin was washed with methylene chloride, dimethylformamide, methylene chloride, methanol, and ether in succession. Thereafter, the washed resin was subjected to vacuum drying. By the PNBP method and the Disperse Red method, it was confirmed that the reaction was progressing but had not yet been completed (coloring reaction was observed by both PNBP and Disperse Red methods) (the uppermost figure in FIG. 1). Acetylation was carried out in order to inactivate unreacted hydroxyl groups. The solid phase resin was suspended in methylene chloride (7 mL), and i-Pr₂NEt (0.6 mL) and Ac₂O (0.3 mL) were dropped in the suspension. The obtained reaction solution was shaken for 2 hours, and the solid phase resin was filtrated. Then, the solid phase resin was washed with methylene chloride, methanol, methylene chloride, and ether in succession, followed by vacuum drying (after acetylation, the development of color was observed by the PNBP method, but the coloring reaction was not observed by the Disperse Red method).

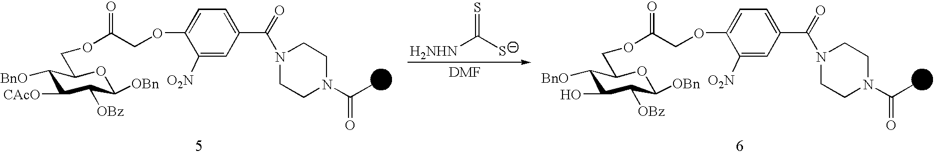

Solid phase resin 5 (1.50 g) was suspended in DMF (10 mL), and a hydrazinedithilocarbonate solution (0.37 M, 2.9 mmol) was dropped in the suspension, followed by shaking at room temperature for 1 hour. The solid phase resin was filtrated and then washed with DMF, methylene chloride, methanol, and ether in succession, Thereafter, the solid phase resin was subjected to vacuum drying (a coloring reaction was not observed by the PNBP method, but it was observed by the Disperse Red method). Thus, from the results of the coloring reaction, it was found that the protection of a chloroacetyl group had been eliminated (deprotected) (the second figure from the top in FIG. 1).

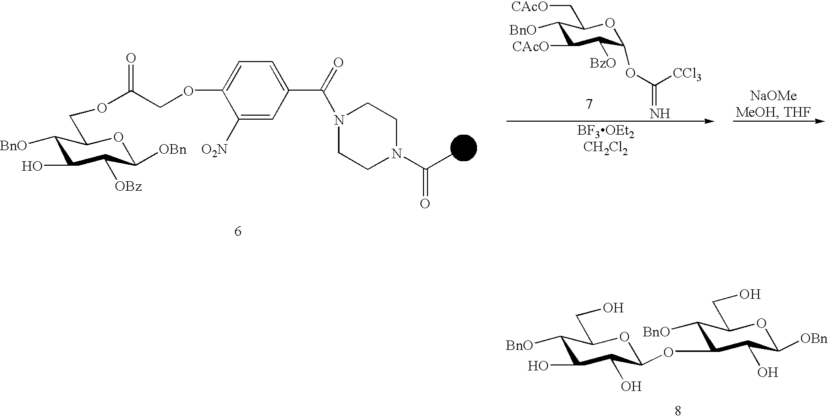

Solid phase resin 6 (1.50 g) and sugar donor 7 were suspended in methylene chloride (10 mL), and BF₃.OEt₂ (0.3 mL) was dropped in the suspension. The reaction solution was shaken at room temperature overnight. The solid phase resin was washed with DMF, methanol, methylene chloride, and ether in succession followed by vacuum drying (a coloring reaction was observed by the PNBP method, but it was not observed by the Disperse Red method) (the third figure from the top in FIG. 1). It was found that a glycosylation reaction had progressed and hydroxyl groups had disappeared.

200 mg out of this solid phase resin was suspended in methanol (3 mL), and Na0Me (0.15 mL, 28% methanol solution) was dropped in the suspension at room temperature. After stirring the mixture overnight, the reaction solution was neutralized with Amberlyst 15E. The solid phase resin was filtrated, and the filtrate was subjected to vacuum concentration. Thereafter, the residue was purified by preparative silica gel thin layer chromatography, so as to obtain compound 8. The disaccharide which was cleaved had a high purity. 1H NMR δ 7.3-7.2(m, 10H), 5.03(d, J=11.2 Hz, 1H), 4.87(d, J=11.7 Hz, 1H), 4.80(d, J=11.2 Hz, 1H), 4.70(d, J=12.4 Hz, 1H), 4.66(d, J=12.0 Hz, 1H), 4.64(d, J=12.4 Hz, 1H), 4.42(d, J=7.6 Hz, 1H), 3.8-3.7(m, 2H), 3.7-3.3(m, 13H), 3.28(m, 1H), 3.27(m, 1H)

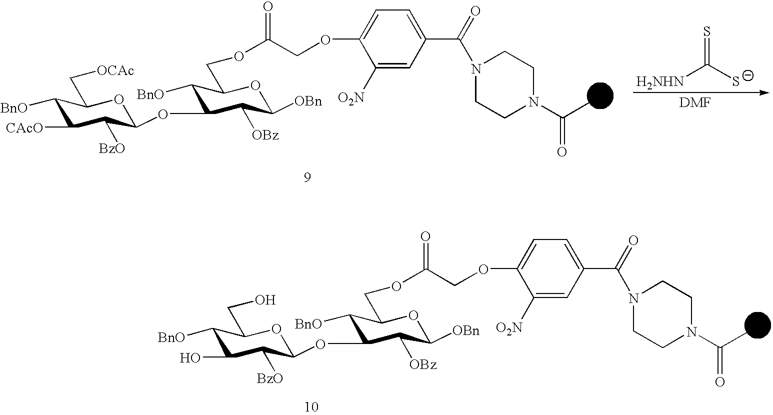

Solid phase resin 9 (500 mg) was suspended in DMF (5 mL), and a hydrazinedithiocarbonate solution (0.37 M, 2.9 mmol) was dropped in the suspension, followed by shaking at room temperature for 1 hour. The solid phase resin was filtrated and then washed with DMF, methylene chloride, methanol, and ether in succession, followed by vacuum drying (a coloring reaction was not observed by PNBP method, but it was observed by Disperse Red method). Thus, it was found that the protection of a chloroacetyl group had been eliminated (deprotected).

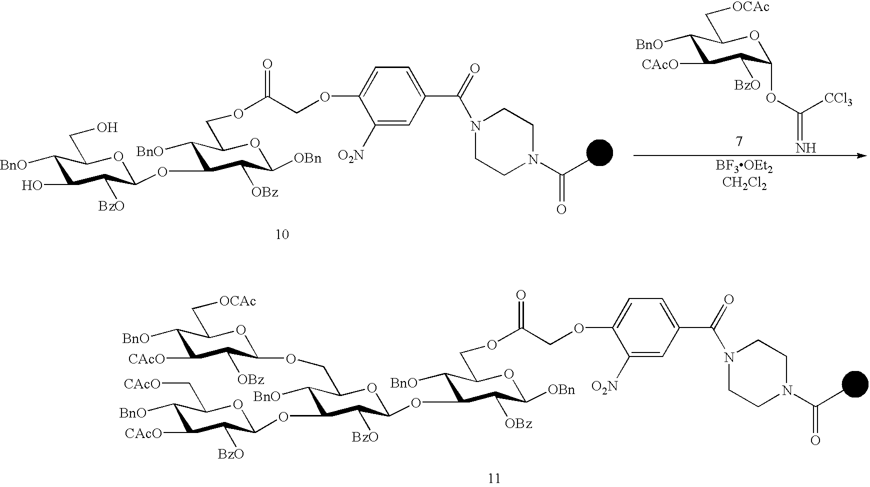

Solid phase resin 10 (500 mg) and sugar donor 7 (464 mg) were suspended in methylene chloride (4 mL), and BF₃.OEt₂ (0.15 mL) was added to the suspension, followed by shaking at room temperature for 13 hours. The solid phase resin was filtrated and then washed with methylene chloride, methanol, DMF, and methylene chloride in succession, followed by vacuum drying (a coloring reaction was observed by the PNBP method, but it was not observed by the Disperse Red method) (the fourth figure from the top in FIG. 1). It was found that hydroxyl groups had disappeared and a glycosylation reaction had progressed.

200 mg out of this solid phase resin was suspended in methanol (3 mL), and Na0Me (0.154 mL, 28methanol solution) was dropped in the suspension at room temperature. After stirring overnight, the reaction solution was neutralized with Amberlyst 15E. The solid phase resin was filtrated, and the filtrate was subjected to vacuum concentration. Thereafter, the residue was purified by preparative silica gel thin layer chromatography, so as to obtain compound 15. The tetraose which was cut out had a high purity,

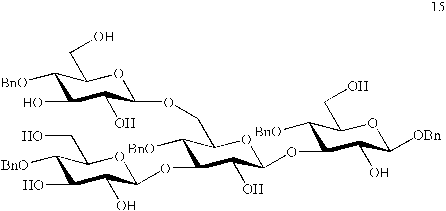

¹H NMR δ (CD₃OD)7.3-7.2(m,25H), 4.99(d,J=10.7 Hz,1H), 4.94(d,J=10.5 Hz,1H), 4.84(d,J=11.0 Hz,1H), 4.82(d,J=11.7 Hz,1H), 4.6-4.5(m,3H),449(d,J=l11.2 Hz, 1H, 4.45(d,J=10.7 Hz,1H), 4.35(d,J=7.8 Hz,1H), 4.31(d,d=7.8 Hz,1H), 4.06(m,1H), 3.9-3.8(m,2H), 3.57(m,1H), 3.6-3.3(m,14H), 3.04(t,J=8.8 Hz,1H), 2.62(m,1H); ¹³C NMR δ 139,98(C),139.88(C),139.70(C),139.42(C),138.91(C),129.56(CH), 129.41(CH),129.13(CH),129.09(CH),129.13(CH),129.09(CH),129.06(CH),129.03(CH),128.9 7(CH),128.83(CH),128.67(CH),128.54(CH),128.49(CH),128.45(CH), 128-34(CH),105.09(CH),104.56(CH),104.09(CH),103.00(CH),85.25(CH),83.70(CH),79.08(C H),78.79(CH),78.39(CH),78.26(CH),77.88(CH),77.78(CH),77.44(CH), 76.75(CH),76.57(CH),76.46(CH),76.09(CH),76.00(CH₂),75.92 (CH₂), 77.72(CH), 75.61 (CH₂),75.47(CH),75.07(CH₂),71.82(CH₂),69.33(CH₂),62.43(CH₂),62.36(CH₂),62.07(C H₂).

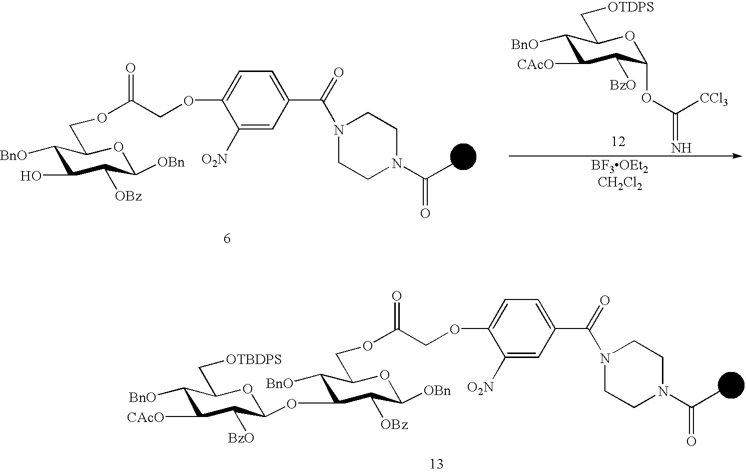

Solid phase resin 6 (200 mg) and sugar donor 12 were suspended in methylene chloride (2 mL), and BF₃.OEt₂ (30 mL) was dropped in the suspension. The reaction solution was shaken for 12 hours, and the solid phase resin was filtered. Then, the solid phase resin was washed with methylene chloride, methanol, DMF, and methylene chloride (a coloring reaction was observed by PNBP method, but it was not observed by Disperse Red method).

### INDUSTRIAL APPLICABILITY

According to the present invention, the progress of a reaction can be simply, quickly and selectively monitored with high sensitivity in real time, in the solid-phase synthesis of a sugar chain.

The present disclosure relates to subject matter contained in priority Japanese Patent Application No. 2002-312131, filed on Oct. 28, 2002, the contents of which is herein expressly incorporated by reference in its entirety.

1. A method for detecting the presence of a hydroxyl group in sugars, which comprises reacting the sugar having a hydroxyl group which is immobilized to a solid phase, with a compound represented by the formula X-Y wherein X represents a residue of an azo dye compound, and Y represents a group capable of reacting with the hydroxyl group of the sugars.

2. A method for detecting the presence of a protected hydroxyl group in sugars, which comprises reacting the sugar having a hydroxyl group protected by a Z—CH₂—CO— group wherein Z represents a halogen or —O—SO₂—R, in which R represents an aliphatic or aromatic hydrocarbon group, which is immobilized to a solid phase, with (p-nitrobenzyl)pyridine under basic conditions.

3. A method for detecting whether or not a hydroxyl group in sugars is protected, which comprises the step of reacting the sugar having a hydroxyl group or hydroxyl group protected by a Z—CH₂—CO— group wherein Z represents a halogen or —O—SO₂—R, in which R represents an aliphatic or aromatic hydrocarbon group, which is immobilized to a solid phase, with a compound represented by the formula X-Y wherein X represents a residue of an azo dye compound, and Y represents a group capable of reacting with the hydroxyl group in the sugars; and/or reacting the above sugar with (p-nitrobenzyl)pyridine under basic conditions.

4. The method of claim 1 wherein the compound represented by the formula X-Y Is N-[2-[(4,6dichloro-1,3,5-triazin-2-yl)oxy]ethyl]-N-ethyl-4-[(4-nitrophenyl)azo]-benzeneamide.

5. The method of claim 3 wherein the compound represented by the formula X-Y is N-[2-[(4,6dichloro-1,3,5-triazin-2-yl)oxy]ethyl]-N-ethyl4[(4-nitrophenyl)azo]-benzeneamide.

6. The method of claim 2 wherein the Z—CH₂—CO— group is a chloroacetyl group.

7. The method of claim 3 wherein the Z—CH₂—CO— group is a chloroacetyl group.

8. A method for monitoring the progress of a synthesis reaction of a sugar chain in the method of synthesizing a sugar chain by reacting the first sugars having a hydroxyl group which is immobilized to a solid phase, with the second sugars having a reactive group reacting with the above hydroxyl group and a protected hydroxyl group, wherein the protecting group of the hydroxyl group is a Z—CH₂—CO— group wherein Z represents a halogen or —O—SO₂—R, in which R represents an aliphatic or aromatic hydrocarbon group, and the presence of a hydroxyl group or a protected hydroxyl group in sugars which is immobilized to a solid phase is detected by the reaction of the sugar with a compound represented by the formula X-Y wherein X represents a residue of an azo dye compound, and Y represents a group capable of reacting with the hydroxyl group in sugars, or (p-nitrobenzyl)pyridine.
